(19) United States
(12) Patent Application Publication
(10) Pub. No.: US 2005/0026977 A1
(43) Pub. Date: Feb. 3, 2005

(54) Zonisamide use in eating disorders

(76) Inventor: Julianne E. Jennings, San Diego, CA (US)

Correspondence Address: Edward D. Grieff;Wilmer Cutler Pickering Hale and Dorr LLP, 1455 Pennsylvania Avenue, NW, Washington, DC, 20004, US

(21) Appl. No.: 10/932,047

(22) Filed: Sep. 2, 2004

Related U.S. Application Data
(63) Continuation of application No. 10/429,474, filed on May 2, 2003.
(60) Provisional application No. 60/378,446, filed on May 6, 2002.

(51) Int. Cl. A61K 31/42

(52) U.S. Cl. 514/379

(57) ABSTRACT

The present invention is directed to a method of treating overweight and/or obesity and eating disorders such as binge-eating disorders, bulimia nervosa and anorexia nervosa. The method comprises administering to a subject a pharmaceutical composition comprising an effective amount of zonisamide (1,2-benzisoxazole-3-methanesulfonamide). The present method provides a sustained and significant weight loss in an overweight subject. The method can be used in conjunction with other therapeutic agents/methods commonly used to treat overweight and eating disorders thus enhancing the therapeutic effect of reducing weight and regulating eating disorders.

### RELATED APPLICATIONS

This application is a continuation of U.S. application Ser. No. 10/429,474 filed May 2, 2003, which claims the priority benefit of U.S. Provisional Application No. 60/378,446, filed May 6, 2002.

### FIELD OF THE INVENTION

The present invention relates to methods of treating overweight, obesity and eating disorders, particularly binge-eating disorders, bulimia nervosa and anorexia nervosa, with zonisamide (1,2-benzisoxazole-3-methanesulfonamide).

### BACKGROUND

Approximately 97 million adults in the United States are estimated to be overweight or obese, with a substantial increase of this epidemic in the recent years. With both conditions, there is a considerable increase in the prevalence of many comorbid illnesses including type 2 diabetes, coronary heart disease, hypertension, gallbladder disease, and osteoarthritis, with an increased risk of mortality from all causes. Significant reduction of obesity-related illnesses and risk factors can occur with a modest (<10%) weight reduction. Although diet, exercise, behavior therapy and pharmacotherapy can be effective, many obese patients fail to achieve significant benefit from any given treatment modality, and the long-term outcome with most non-surgical treatments is often unsatisfactory. Currently drugs available for treating obesity include XENICAL® (Orlistat), MERIDIA®, and amphetamines. XENICAL® is a lipase inhibitor that reduces dietary fat absorption; but has gastrointestinal side effects related to oil elimination. MERIDIA® (sibutramine hydrochloride) is a serotonin, norepinephrine and to a lesser extent dopamine re-uptake inhibitor; its side effects include dry mouth, anorexia, insomnia, constipation and headache. MERIDIA® is recommended for obese patients with an initial body mass index >/=30 kg/m², or >/=27 kg/m² in the presence of other risk factors (e.g., hypertension, diabetes, dyslipidemia). Amphetamines such as methamiphetamine, phentermine and phendimetrazine are only prescribable for short-term treatment.

It is estimated that anywhere from 2 to 10% of Americans have eating disorders. The biology involved in eating disorders is very complex and not well understood. The goal of treatment is to normalize eating behavior. PROZAC® or fluoxetine is currently the only agent approved for the treatment of bulimia nervosa. TOPAMAX® (topiramate/TPM) has been shown in preliminary studies to suppress the appetite and to reduce the amount of binging. Patients on TPM have also reported loss of hunger and preoccupation with food.

There is a need for a method to treat an overweight and/or obese subject such that the weight loss is significant and sustained over time. There is also a need for a method to treat eating disorders such as binge-eating disorders, bulimia nervosa and anorexia nervosa.

Zonisamide is an antiseizure drug classified as a sulfonamide and chemically unrelated to other antiseizure agents. Zonisamide has the chemical structure of 1,2-benzisoxazole-3-methanesulfonamide and is further characterized in the Merck Index (11^{th} Ed. 1989) at monograph no. 10094. Zonisamide and related structures are described in described in U.S. Pat. No. 4,172,896, which is hereby incorporated herein by reference in its entirety for all purposes. It is approved for use in humans in the United States and in Japan. The mechanism(s) by which zonisamide exerts its antiseizure activity is unknown. Zonisamide has demonstrated an anticonvulsant activity in threshold for generalized seizures in the kindled rat model. Zonisamide has reduced the duration of cortical focal seizures induced by electrical stimulation of the visual cortex in cats. Furthermore, zonisamide suppresses both interictal spikes and the secondarily generalized seizures produced by cortical application of tungstic acid gel in rats or by cortical freezing in cats.

Walker, et al. (Fundam Appl. Toxicol., 11:333-42 (1988)) disclose that when testing chronic toxicity of zonisamide in beagle dogs, early body weight losses occurred in dogs given 75 mg/kg/day. Morris (Epilepsia, 41: 39 (2000)) discloses that weight loss was an adverse event for patients treated with anti-epilepsy drugs zonisamide, however, the weight loss did not continue over time. Ginsberg, et al. (Primary Psychiatry, 7: 49-58 (2000)) reported loss of appetite as an adverse effect of zonisamide, an antimanic agent. Ginsberg, et al. also suggest a potential role for zonisamide in the management of psychotropic-induced weight gain.

Zonisamide may produce anti-epileptic and anti-convulsant effects through action at sodium and calcium channels. In vitro pharmacological studies suggest that zonisamide blocks sodium channels and reduces voltage-dependent, transient inward currents (T-type Ca²⁺ currents), consequently stabilizing neuronal membranes and suppressing neuronal hypersynchronization. In vitro binding studies have demonstrated that zonisamide binds to the GABA/benzodiazepine receptor ionophore complex in an allosteric fashion which does not produce changes in chloride flux. Other in vitro studies have demonstrated that zonisamide (10-30 μg/mL) suppresses synaptically-driven electrical activity without affecting postsynaptic GABA or glutamate responses (cultured mouse spinal cord neurons) or neuronal or glial uptake of [^{3H}]-GABA (rat hippocampal slices). Thus, zonisamide does not appear to potentiate the synaptic activity of GABA. In vivo microdialysis studies have demonstrated that zonisamide facilitates both dopaminergic and serotonergic neurotransmission. Based on the effects such as blocking sodium channels and reducing voltage-dependent, transient inward currents (T-type Ca²⁺ currents), modulation of serotonergic and dopaminergic neurotransmission, Applicants discovered that zonisamide is efficacious in treating overweight, obesity, and eating disorders.

### SUMMARY OF THE INVENTION

The present invention is directed to a method of reducing weight in an overweight and/or obese subject, wherein said weight loss is significant and sustained . The method comprises administering to a subject a pharmaceutical composition comprising zonisamide, in an amount effective to reduce weight or to induce weight loss in said subject wherein the weight loss or the induction of weight loss is sustained during the dosing regimen.

The invention is also directed to a method of treating eating disorders in a subject in need of such treatment, to alleviate the symptoms of eating disorders. The method comprises administering to a subject a pharmaceutical composition comprising zonisamide, in an amount effective to suppress appetite or stimulate the satiety reflex in the subject.

The pharmaceutical composition can be administered in the range of 50 to 600 mg per day through a variety of routes of administration, including oral, topical, rectal, injection, or implantation. A preferred route of administration is via oral dosing.

### DETAILED DESCRIPTION OF THE INVENTION

Zonisamide has several pharmacologic actions important in obesity and eating disorders including carbonic anhydrase inhibition as well as dopaminergic and serotonergic neurotransmission. The pharmacokinetic and drug interaction profiles of zonisamide are ideal for treating patients with obesity or eating disorders.

The present invention provides a method of reducing weight in an overweight and/or obese subject. The method comprises administering to the subject a pharmaceutical composition comprising an effective amount of zonisamide to reduce weight in a subject such that the weight loss is significant and sustained. Alternatively, the method comprises administering to the subject a pharmaceutical composition comprising zonisamide in an amount effective to induce weight loss in said subject, wherein the induction of weight loss is sustained during the dosing regimen.

The present method is useful in treating the overweight and/or obese population. Overweight refers to an excess of body weight compared to set standards. The excess weight may come from muscle, bone, fat, and/or body weight. Obesity refers specifically to having an abnormally high proportion of body fat. One can be overweight without being obese, as in the example of the body builder or other athlete who has a lot of muscle. However, many people who are overweight are also obese. Except for heavily muscled persons, a body weight 20% over that in standard height-weight tables is arbitrarily considered obesity. Obesity may be classified as mild (20 to 40% overweight), moderate (41 to 100% overweight), or severe (>100% overweight). The National Institutes of Health (NIH) identify overweight as a body mass index (BMI) of 25-29.9 kg/m² and obesity as a BMI of 30 kg/m² or greater. (National Institutes of Health/National Institute of Diabetes & Digestive & Kidney Diseases; page 981 in “Nutritional and Metabolic Disorders” in The Merck Manual of Diagnosis and Therapy, 16th Edition, 1992). BMI is calculated by taking a subject's weight, in kg, divided by the subject's height, in meters, and squared. Table 1 shows a chart of BMI based on various heights and weights. The present method is effective in reducing weight in mild, moderate and severe obese subjects.

TABLE 1
BMI Chart

| | | 25 | 26 | 27 | 28 | 29 | 30 | 31 | 32 | 33 | 34 | 35 | 40 |
|---|---|---|---|---|---|---|---|---|---|---|---|---|---|
| BMI | | WEIGHT (lbs) | | | | | | | | | | | |
| HEIGHT | 4′10″ | 119 | 124 | 129 | 134 | 138 | 143 | 149 | 153 | 158 | 163 | 167 | 191 |
| | 4′11″ | 124 | 128 | 133 | 138 | 143 | 148 | 154 | 158 | 164 | 169 | 173 | 198 |
| | 5′ | 128 | 133 | 138 | 143 | 148 | 153 | 159 | 164 | 169 | 175 | 179 | 204 |
| | 5′1″ | 132 | 137 | 143 | 148 | 153 | 158 | 165 | 169 | 175 | 180 | 185 | 211 |
| | 5′2″ | 136 | 142 | 147 | 153 | 158 | 164 | 170 | 175 | 181 | 186 | 191 | 218 |
| | 5′3″ | 141 | 146 | 152 | 158 | 163 | 169 | 175 | 181 | 187 | 192 | 197 | 225 |
| | 5′4″ | 145 | 151 | 157 | 163 | 169 | 174 | 181 | 187 | 193 | 199 | 204 | 232 |
| | 5′5″ | 150 | 156 | 162 | 168 | 174 | 180 | 187 | 193 | 199 | 205 | 210 | 240 |
| | 5′6″ | 155 | 161 | 167 | 173 | 179 | 186 | 192 | 199 | 205 | 211 | 216 | 247 |
| | 5′7″ | 159 | 166 | 172 | 178 | 185 | 191 | 198 | 205 | 211 | 218 | 223 | 255 |
| | 5′8″ | 164 | 171 | 177 | 184 | 190 | 197 | 204 | 211 | 218 | 224 | 230 | 262 |
| | 5′9″ | 169 | 176 | 182 | 189 | 196 | 203 | 210 | 217 | 224 | 231 | 236 | 270 |
| | 5′10″ | 174 | 181 | 188 | 195 | 202 | 207 | 216 | 223 | 230 | 237 | 243 | 278 |
| | 5′11″ | 179 | 186 | 193 | 200 | 208 | 215 | 222 | 230 | 237 | 244 | 250 | 286 |
| | 6′ | 184 | 191 | 199 | 206 | 213 | 221 | 228 | 236 | 244 | 251 | 258 | 294 |
| | 6′1″ | 189 | 197 | 204 | 212 | 219 | 227 | 236 | 243 | 251 | 258 | 265 | 302 |
| | 6′2″ | 194 | 202 | 210 | 218 | 225 | 233 | 241 | 250 | 258 | 265 | 272 | 311 |
| | 6′3″ | 200 | 208 | 216 | 224 | 232 | 240 | 248 | 256 | 264 | 272 | 279 | 319 |

The present invention is advantageous in that it results a significant and sustained weight loss in an overweight and/or obese subject. A significant weight loss means that a subject loses greater than or equal to 3%, preferably 5%, more preferably 7%, and most preferably 10% of body weight. A sustained weight loss means that the weight loss in a subject does not plateau after the subject has lost a few pounds of weight. Patients treated by the present method continue to lose weight as long as they remain on the zonisamide treatment.

The present invention is also directed to a method of treating eating disorders, including but not limited to binge-eating disorders, bulimia nervosa and anorexia nervosa, in a subject in need of such treatment. The method comprises administering to a subject a pharmaceutical composition comprising zonisamide, or its pharmaceutically acceptable salt thereof, in an amount effective to suppress appetite or stimulate the satiety reflex in the subject, such that the symptoms of eating disorders are alleviated.

Binge-eating disorder is classified as an eating disorder not otherwise specified in the Diagnostic and Statistical Manual of Mental Disorders, Fourth Edition (DSM-IV) such as recurrent episodes of binge eating associated with the regular use of inappropriate compensatory behaviors (e.g., purging, fasting, excessive exercise). Binge-eating disorder is common among obese individuals (body mass index [BMI]>30 kg/m²) seeking treatment, occurring in approximately 30% of obese individuals in weight-loss treatment programs and 70% of individuals in Overeaters Anonymous. It may also be common among the general population.

Bulimia is an emotional disorder characterized by episodes of binge eating followed by a method of purge at least two days per week for a period of at least three months. During the episodes, the person eats to control overpowering emotions, and is not usually hungry. The purge can be any of the following methods: vomiting, laxatives, diet pills, over exercising, diuretics, and/or fasting. Sometimes the persons use more than one method of purging. Although predominantly a female disorder, many males are also affected. Bulimia may start out as a simple diet, escalating into a binge/purge cycle like an addiction.

Anorexia nervosa is a serious, potentially life-threatening eating disorder characterized by self-starvation and excessive weight loss. Anorexia nervosa is an emotional disorder characterized by an intense fear of fat that results in extreme dieting. Anorexia nervosa is characterized by loss of at least 10% of normal body weight or a failure to reach within 10% of the normally expected weight. It affects mostly girls and women between the ages of 12 and 24. Sometimes older women, and boys or men have also been known to struggle with anorexia nervosa. Low self-esteem, distorted body image, and an obsession with food are other distinguishing features. This relentless pursuit of thinness results in death in as many as 10 to 15% afflicted with the disorder.

The present invention provides a method for regulating eating behaviors by reduction of hunger, suppression of appetite, loss of preoccupation with food, and/or enhancement of satiety. The method treats patients with eating disorders such that they stop binging or only binge small amounts.

The pharmaceutical formulation of the present invention can be applied by any of the accepted modes of administration for agents which affect the central nervous system (CNS) including oral, parenteral, topical, injection, rectal, and other routes of administration. Any pharmaceutically acceptable mode of administration can be used, including solid, semi-solid, or liquid dosage forms, such as for example, tablets, suppositories, pills, capsules, powders, liquids suspensions, or the like, preferably in unit dosage form suitable to single administration of precise dosages, or in sustained or controlled release forms for the prolonged administration of the compound at a predetermined rate. The compositions will typically include a conventional pharmaceutical carrier or excipient and the drug product zonisamide and, in addition, may include other medicinal agents, pharmaceutical agents, carriers, etc. The compositions are advantageously compounded into unit dosage forms, containing a predetermined, standard amount of the active compound, to make dosing and patient compliance simpler.

The amount of active compound administered will be dependent on the subject being treated, the severity of the affliction, the manner of administration and the judgment of the prescribing physician. In general, an effective dosage is in the range of 50-1000 mg/day, preferably 100-600 mg/day, which may be administered all at a time or in divided doses. The dosage of these compounds may vary in accordance with the administration route, the age and the body weight of the patient and the degree of the therapeutic effect desired. A higher amount may be administered to a patient with higher body weight.

The compounds of the present invention are usually administered in the form of a pharmaceutical composition which contains them in admixture with a pharmaceutical carrier. The pharmaceutical composition may be in the dosage forms such as tablets, capsules, granules, fine granules, powders, syrups, suppositories, injections, or the like. These preparations can be prepared by conventional methods.

The carriers useful for these preparations include all organic or inorganic carrier materials which are usually used for the pharmaceutical preparations and are inert to the active ingredient. Examples of the carriers suitable for the preparation of tablets capsules, granules and fine granules are diluents such as lactose, starch, sucrose, D-mannitol, calcium sulfate, or microcrystalline cellulose; disintegrators such as sodium carboxymethylcellulose, modified starch, or calcium carboxymethylcellulose; binders such as methylcellulose, gelatin, acacia, ethylcellulose, hydroxypropylcellulose, or polyvinylpyrrolidone; lubricants such as light anhydrous silicic acid, magnesium stearate, talc, or hydrogenated oil; or the like. When formed into tablets, they may be coated in a conventional manner by using conventional coating agents such as calcium phosphate, carnauba wax, hydroxypropyl methylcellulose, macrogol, hydroxypropyl methylphthalate, cellulose acetate phthalate, titanium dioxide, sorbitan fatty acid ester, or the like.

Examples of carriers suitable for the preparation of syrups are sweetening agents such as sucrose, glucose, fructose, or D-sorbitol; suspending agents such as acacia, tragacanth, sodium carboxymethylcellulose, methylcellulose, sodium alginate, microcrystalline cellulose, or veegum; dispersing agents such as sorbitan fatty acid ester, sodium lauryl sulfate, or polysorbate 80; or the like. When formed into syrups, the conventional flavoring agents, aromatic substances, preservatives, or the like may optionally be added thereto. The syrups may be in the form of a dry syrup which is dissolved or suspended before use.

Examples of bases used for the preparation of suppositories are cacao butter, glycerinsaturated fatty acid ester, glycerogelatin, macrogol, or the like. When formed into suppositories, the conventional surface active agents, preservatives or the like may optionally be admixed.

When formed into injections, the alkali metal salt of the compound is dissolved in distilled water for injection, to which may optionally be added the conventional solubilizers, buffering or pH adjusting agents, isotonic agents, preservatives and other suitable substances. The injections may be in the solid dry preparations which are dissolved before use.

These pharmaceutical compositions usually contain zonisamide or its alkali metal salt as the active ingredient in an amount of 0.5% by weight or more, preferably 10 to 70% by weight, based on the total weight of the composition. These compositions may optionally contain other therapeutically active compounds.

For solid compositions, conventional non-toxic carriers include, for example mannitol, lactose, starch, magnesium stearate, sodium saccharin, talcum, cellulose, glucose, sucrose, magnesium carbonate, and the like may be used. The active compound as defined above may be formulated as suppositories using, for example, polyalkylene glycols, for example, propylene glycol as a carrier. Liquid pharmaceutically administrable compositions can, for example, be prepared by dissolving, dispersing, etc. an active compound as defined above and optional pharmaceutical adjuvants in a carrier, such as, for example, water, saline, aqueous dextrose, glycerol, ethanol, and the like to thereby form a solution or suspension. If desired, the pharmaceutical composition to be administered may also contain minor amounts of non-toxic auxiliary pH buffering agents and the like, for example, sodium acetate, sorbitan monolaurate, triethanolamine oleate, etc. Actual methods of preparing such dosage forms are known, or will be apparent to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount effective to alleviate the symptoms of the subject being treated.

Dosage forms or compositions containing active ingredient of zonisamide in the range of 0.25 to 95% with the balance made up from non-toxic carrier may be prepared. For oral administration, a pharmaceutically acceptable non-toxic composition is formed by the incorporation of any of the normally employed excipients, and may contain 1%-95% active ingredient, preferably 5%-50%.

Parenteral administration is generally characterized by injection, whether subcutaneously, intramuscularly, or perineurally. Injectables can be prepared in conventional forms, either as liquid solutions or suspension, solid forms suitable for solution or suspension in liquid prior to injection, or as emulsions. Suitable excipients include, for example, water, saline, aqueous dextrose, glycerol, ethanol or the like. In addition, if desired, the pharmaceutical compositions may also contain minor amounts of non-toxic substances such as wetting or emulsifying agents, auxiliary pH buffering agents and the like, for example, sodium acetate, sorbitan monolaurate, triethanolamine oleate, etc.

The percentage of active compound contained in such parenteral compositions is highly dependent on the specific nature and the activity of zonisamide and the needs of the subject, However, percentages of active ingredient of 0.1% to 10% in solution are employable, and is higher if the composition is a solid which will be subsequently diluted to the above percentages. Preferably the composition comprises 0.2-2% of the active agent in solution.

Other modes of administration can also be practiced in accordance with the present invention. For example, intravenous, intramuscular, and subcutaneous delivery are examples of delivery methods that are contemplated by the present invention.

For delayed release, the compounds of the invention may be included in a pharmaceutical composition for formulated for slow release, such as in microcapsules formed from biocompatible polymers or in liposomal carrier systems according to methods known in the art.

For continuous release of active agent, zonisamide may be covalently conjugated to a water soluble polymer, such as a polylactide or biodegradable hydrogel derived from an amphipathic block copolymer, as described in U.S. Pat. No. 5,320,840. Collagen-based matrix implants, such as described in U.S. Pat. No. 5,024,841, are also useful for sustained delivery of therapeutics.

The method of the present invention can be used with other therapeutic agents/methods commonly used to treat obesity or eating disorders, thus enhancing the effects of therapeutic agents and therapeutic methods. Other therapeutic agents/methods used for treating obesity include hypocaloric diets, exercise, orlistat, amphetamines (methamphetamine, phentermine and phendimetrazine), sibutramine, and topiramate. Other therapeutic agents/methods used for treating eating disorders include fluoxetine and topiramate.

High doses are sometimes required for some therapeutic agents to achieve levels to effectuate the target response, but high doses often associate with a greater frequency of dose-related adverse effects. Thus, combined use of the pharmaceutical composition of the present invention with therapeutic agents commonly used to treat obesity or eating disorders allows the use of relatively lower doses of other agents, which results in a lower frequency of adverse side effects associated with long-term administration of such agents. Thus, another advantage of the compounds in this invention is to reduce adverse side effects of drugs used to treat obesity or eating disorders, such as tolerance, dependence, constipation, respiratory depression, sedation, and gastrointestinal side effects.

### EXAMPLE

The following examples further illustrate the present invention. These examples are intended merely to be illustrative of the present invention and are not to be construed as being limiting.

### Example 1

### Zonisamide in Obesity: A 16-Week Randomized Controlled Trial

Objective: Short-term efficacy and safety of zonisamide in the treatment of obesity was evaluated.

Study Population

Inclusion Criteria: (1) Age 21-50; (2) BMI of 30-44 kg/m²; and (3) Otherwise healthy as determined by the principal investigator.

Exclusion Criteria: (1) Obesity of known endocrine etiology, e.g., hypothyroidism, Cushing's syndrome, polycystic ovarian disease, etc.; (2) Serious or unstable illness, e.g., significant cardiovascular disease, history of stroke, epilepsy, etc.; (3) History of renal calculi; (4) Significant hepatic or renal disease; (5) Uncontrolled HTN; Current Type I diabetes mellitus or Type II DM on pharmacotherapy; (6) Untreated or uncontrolled thyroid disease; (7) Current use of other weight loss medications; (8) Weight loss of >4 kg in the past three months; (9) Had surgery for obesity; (10) Current major psychiatric disorder; (11) Current alcohol or drug abuse; (12) Current or recent use of medications that have the potential to compromise study safety, or pose difficulties in interpreting the study outcomes, e.g., medications known to significantly affect body weight; (13) Current use of medications that significantly induce or inhibit P450 3A4 hepatic enzymes; (14) Allergy or hypersensitivity to sulfonamides; (15) Women of child-bearing potential, not adhering to an acceptable form of contraception; (16) Pregnant or breast-feeding women; and (17) Subjects, judged to be inappropriate by the principal investigator, for other reasons such as risk of non-compliance or inability to follow study procedures.

Method: 60 subjects were assigned to receive zonisamide or placebo (1:1 ratio) in a randomized, double-blind fashion for 16 weeks in addition to a slightly hypocaloric (500 kcal/day deficit) diet. Zonisamide dosing was flexible with a maximum of 600 mg/day.

Study Drug and Dosing

Zonisamide was started at 100 mg/d. Based on tolerability, the dose was titrated up as follows: Weeks 3-4: 200 mg/d; Weeks 5-6: 300 mg/d; Weeks 7-12: 400 mg/d.

At the end of week 12, for those where at least 5% weight loss was not achieved, the dose was further increased to 600 mg/d.

The entire dose is given at bedtime. However, based on tolerability, a part of the daily dose may be given in the morning.

Results: Using the available data for all randomized subjects with the last observation carried forward, the zonisamide group lost, on average, more bodyweight than the placebo group (5.98% vs. 1.09%; p<0.0001) during the 16-week period. 17/30 subjects in the zonisamide group and 3/30 in the placebo group lost .gtoreq.5% weight (p<0.0003). A random coefficient regression for weight change, with effects for age, race, gender, BMI, and percent body fat, estimated that zonisamide treatment over the 16-week study duration was associated with a 4.99 kg greater weight loss over placebo treatment (p<0.0001). Zonisamide was tolerated well with minimal side effects.

Conclusion: Zonisamide was significantly more effective than placebo as an adjunct to hypocaloric diet in the treatment of obesity.

Although the invention has been described with reference to the presently preferred embodiments, it should be understood that various modifications can be made without departing from the scope of the invention.

1. A method for treating a binge eating disorder in a patient in need thereof comprising administering zonisamide or a pharmaceutically acceptable salt thereof in an amount of about 50 mg/day to about 1,000 mg/day.

2. A method for treating bulimia in a patient in need thereof comprising administering zonisamide or a pharmaceutically acceptable salt thereof in an amount of about 50 mg/day to about 1,000 mg/day.

3. A method for treating anorexia nervosa in a patient in need thereof comprising administering zonisamide or a pharmaceutically acceptable salt thereof in an amount of about 50 mg/day to about 1,000 mg/day.

4. A method for treating an eating disorder in a patient in need thereof comprising administering a therapeutically effective amount of zonisamide or a pharmaceutically acceptable salt thereof.

5. The method of claim 4, wherein the eating disorder is a binge eating disorder.

6. The method of claim 4, wherein the eating disorder is bulimia nervosa.

7. The method of claim 4, wherein the eating disorder is anorexia nervosa.

8. The method of claim 4, wherein the zonisamide or the pharmaceutically acceptable salt thereof is administered in an amount of about 50 to about 1000 mg/day.

9. The method of claim 4, wherein the zonisamide or the pharmaceutically acceptable salt thereof is administered in an amount of about 100 to about 600 mg/day.

10. The method of claim 4, wherein zonisamide or the pharmaceutically acceptable salt thereof is administered parenterally, topically or rectally.

11. The method of claim 4, wherein zonisamide or the pharmaceutically acceptable salt thereof is administered orally.

12. The method according of claim 4, wherein the method further comprises administering another therapeutic agent used to treat an eating disorder.

13. The of claim 4, further comprising administering a therapeutically effective amount of fluoxetine, topiramate or orlistat.
